(19) United States
(12) Patent Application Publication
(10) Pub. No.: US 2007/0060898 A1
(43) Pub. Date: Mar. 15, 2007

(54) Enteral medical treatment assembly having a safeguard against erroneous connection with an intravascular treatment system

(76) Inventors: Michael C. Shaughnessy, Arlington Heights, IL (US); Shawn G. Purnell, Palatine, IL (US)

Correspondence Address: Bell, Boyd & Lloyd LLC, P.O. Box 1135, Chicago, IL, 60690-1135, US

(21) Appl. No.: 11/223,868

(22) Filed: Sep. 7, 2005

(51) Int. Cl. A61M 25/00

(52) U.S. Cl. 604/284; 604/910; 604/523

(57) ABSTRACT

An enteral medical treatment assembly having an enteral supply tube and an enteral access device. The distal end of the enteral supply tube has a mating obstructer configured to obstruct mating of the enteral supply tube with an intravascular patient access port. The proximal end of the enteral patient access device includes a mating obstructer configured to obstruct mating of the enteral patient access device with a connector of an intravascular supply tube. This enteral medical treatment assembly provides a safeguard against erroneous connections between enteral and intravascular treatment systems.

### BACKGROUND

Enteral nutrition involves a form of hyperalimentation and metabolic support in which nutrient formula or medicine is delivered to the gastrointestinal tract, either the stomach or the duodenum. Nutrient administration can be accomplished through use of an enteral feeding system, assembly or device. Enteral feeding systems frequently use catheters inserted through the patient's nose or mouth for administering nutrients to the gastrointestinal tract.

Other types of catheters are inserted into the patient's veins or arteries for intravascular treatment which involves treating the circulatory or cardiovascular system by accessing any blood vessel. These catheters include intravenous (IV) catheters insertable into veins and intra-arterial catheters insertable into arteries. The IV catheters include, among others, the central venous catheter, peripheral venous catheter and the peripherally inserted central catheter (PICC). These catheters include a relatively small tube that passes through the patient's veins. The health care provider uses these catheters to provide patients with injections of medications, drugs, fluids, nutrients, or blood products over a period of time, typically several weeks or more.

When physicians and nurses perform medical care on a patient, especially a pediatric or neonatal patient, it is common that the patient may require both IV treatment as well as enteral nutrition or medicinal supplements. In general, IV assemblies and enteral feeding assemblies include a variety of different fluid sources, tubes and connectors. It is possible that a physician or a nurse may accidentally connect a connector from an enteral feeding system to a connector of an IV system. Therefore, there is a risk that enteral fluid intended for the patient's digestive system may be injected into the patients' blood stream. Similarly, there is a risk that IV fluid may accidentally be introduced into the gastrointestinal tract. This presents a health risk to patients. Therefore, there is a need to overcome the disadvantages described above or otherwise lessen the effects of such disadvantages.

### SUMMARY

The present disclosure generally relates to an assembly for an enteral medical treatment system. The assembly, in one embodiment, includes one or more IV blocking members, to prevent or reduce the likelihood of a potential misconnection of the enteral fluid source to the connectors of an IV access port or intra-arterial access port connected to the patient's vein or artery.

The enteral medical treatment assembly, in one embodiment, includes a patient feeding source such as a gravity bag or pump driven syringe. The feeding source is connected, directly or indirectly, through a tubing set to a feeding tube or an enteral delivery tube. The enteral delivery tube includes a Y-port connector or a multi-branched connector at one end. One of the branches includes a male connector that is matable to the enteral feeding source, and the second branch includes a female connector that is matable to any of variety of other suitable enteral fluid sources. Both the male connector and the female connector of the Y-port connector include IV blocking members that obstruct mating of the Y-port connector with an IV connector, such as an IV male luer lock connector. In one embodiment, the female connector blocking member is structurally different from the male connector blocking member.

In one embodiment, the enteral medical treatment assembly includes an enteral administration tube, where one end of the enteral administration tube is fluidly connected to a patient feeding source, such as a gravity bag or pump driven syringe. The other end of the enteral administration tube includes a female connector. The female connector includes an IV blocking member that obstructs mating of the female connector with an IV access port, such as an IV female luer lock connector connected to the patient's vein or artery.

In operation of one embodiment, a user enterally feeds a patient by delivering medicated or non-medicated feeding fluid from a feeding source through an enteral supply tube, and through an enteral access device connected to the patient. The connector which connects the enteral supply tube to the enteral access device includes an IV obstructer or blocking member. Also, the ports of the Y-port connector each incorporate an IV obstructer. Depending upon the embodiment, the IV blocking members or IV obstructers provide one or more of the following safeguards: (a) obstruction of an erroneous connection of an enteral fluid source to one or more portions of an intravascular treatment system; and (b) obstruction of an erroneous connection of an one or more portions of an intravascular treatment system to an enteral access port.

Additional features and advantages are described herein, and will be apparent from, the following Detailed Description and the figures.

### BRIEF DESCRIPTION OF THE FIGURES

FIG. 1 is a perspective view of the enteral feeding assembly being used to administer fluids to a patient in one embodiment.

FIG. 2 is a perspective view of the enteral feeding assembly in a connected state in one embodiment.

FIG. 3 is a perspective view of the enteral feeding assembly in an unconnected state in one embodiment.

FIG. 4 is a perspective view of an attempted erroneous connection between a supply portion of the enteral feeding assembly and a delivery portion of an intravenous assembly in one embodiment.

FIG. 5 is an enlarged perspective view of an attempted erroneous connection between a supply portion of the enteral feeding assembly and a delivery portion of an intravenous assembly in one embodiment.

FIG. 6 is an enlarged perspective view of an attempted erroneous connection between a supply portion of an intravenous assembly and the delivery portion of the enteral feeding assembly in one embodiment.

FIG. 7 is a cross-section view of the second female fitting of the enteral feeding assembly in one embodiment.

FIG. 8 is a cross-section view of the y-shaped manifold of the enteral feeding assembly in one embodiment.

FIG. 9 is an enlarged perspective view of a connection between the second female fitting and the y-shaped manifold of the enteral feeding assembly in one embodiment.

### DETAILED DESCRIPTION

1. Side-By-Side Enteral and IV Treatment

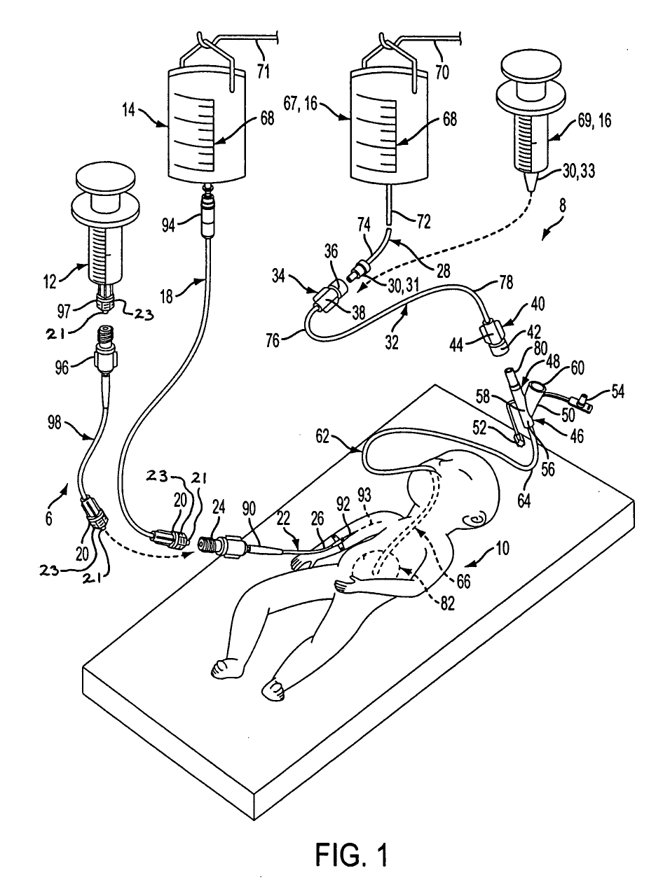

Referring now to the drawings, FIG. 1 illustrates one embodiment of an enteral feeding unit or assembly 8 positioned adjacent to an intravenous administration set, intravenous supply tube assembly or intravenous assembly 6. In the example embodiment, the patient 10 is supplied with both an enteral feeding solution and an intravenous solution. However, it should be appreciated that the patient may be supplied with an enteral feeding solution at the same or at different times than the IV solution. From time to time, the health care provider may disconnect either the IV fluid supply or the enteral fluid supply to introduce a new feeding bag or medicinal syringe. Using the enteral assembly 8 aside the IV assembly 6 enables the health care provider to intravenously administer medicine at certain times of the day or for certain durations while enterally delivering a feeding formula at certain times of the day or for certain durations. The enteral feeding solution may include liquid food, medicine, nutritional supplements or any suitable combinations of the foregoing, while the IV solution can include medication, drugs, fluids or blood products.

1.1 Enteral Medical Treatment Assembly

The enteral medical treatment assembly or feeding assembly 8, in one embodiment, includes: (a) an enteral feeding bag, container or source 16 connected to an enteral supply tube or enteral administration tube 28; (b) an enteral supply tube 32 connected to the enteral administration tube 28; and (c) a feeding tube, catheter or an enteral access tube or enteral access device 62 connected to a multi-way connector, multi-branch connector or Y-port connector 46.

1.1.1 Enteral Feeding Source

In one embodiment, the enteral feeding source 16 includes a container having a plurality of volumetric markings 68 which enable the user to measure the amount of enteral solution that has been administered to the patient 10. The enteral feeding container 16 can include any suitable type of fluid container, such as fluid bag 67 or a syringe 69 or other suitable holder. The markings 68 can include or incorporate any suitable marking or marking system. In one embodiment, the markings 68 are lines printed or painted on the enteral feeding bag 16. In another embodiment, the markings 68 are suitable decals affixed to the enteral feeding bag 16. In one embodiment, the markings 68 are suitable impressions, engravings, grooves or other shape modifications to the enteral feeding bag 16 which a user can use to measure the volume of fluid dispensed. It should be appreciated that the enteral feeding assembly 8 can include markings for fluid measurement purposes on the administration tube 28, enteral supply tube 32, or enteral access device 62.

The enteral feeding source 16 is supported by a support or hook 70 of a stand or other structure. In operation, the feeding source 16 contains a supply of enteral or feeding fluid, and either gravity or a pump device (not shown) forces the feeding fluid from the feeding source 16 through the enteral administration tube 28. In one embodiment, the enteral connector 30 may be integrally attached to the feeding bag 16. In another embodiment, the enteral administration tube 28 and the enteral connector 30 are separate from the feeding bag 16, but may be fluidly connected to the feeding bag by piercing or spiking a proximal end 72 of the enteral administration tube 28 through a portion of the feeding bag 16. In one embodiment, the enteral administration tube 28 includes a step-shaped male connector 31 connected to the distal end 74 of the administration tube 28, and the proximal end 72 is fluidly connected to the enteral feeding source 16. In one embodiment, where the enteral feeding source 16 is a syringe 69, the syringe includes an integrally mounted tapered connector 33, and fluid flows directly from the syringe 69 through the tapered connector 33. In general, the enteral connector 30, unlike a luer connector for an IV system, can include an oral tip connector or any other suitable connector having a tapered or stepped shape.

1.1.2 Enteral Supply Tube and Enteral Access Device

In one embodiment, the enteral supply tube 32 includes a proximal female connector 34 connected to the proximal end 76 of the enteral supply tube 32, and a distal female connector 40 connected to the distal end 78 of the enteral supply tube 32. As illustrated in FIG. 1, the female connector 34 is configured to receive the enteral connector 30, and these connectors 34 and 30 are removably secured to each other through a press-fit connection. The female connector 34 and the female connector 40 include lumens 38 and 44, respectively, allowing fluid to pass from the female connectors 34 and 40 through the enteral supply tube 32. The female connector 34 includes a mating port 36 adapted to receive the enteral connector 30 of the enteral administration tube 28. The female connector 40 includes an identical mating port 42, though it should be appreciated that the ports 36 and 42 can have different shapes in other embodiments. In the embodiment where the ports 36 and 42 are identical, it is possible that both the proximal female connector 34 or the distal female connector 40 can be connected to the enteral connector 30. To assist users in tracking which of the female connectors 34 and 40 is to be connected to the administration tube 28, the distal female connector 40 has a different color than the proximal female connector 34. Also, the color of the distal female connector 40 matches the color of the male connector 80 described below. This color scheme indicates to the user which end of the enteral supply tube 32 should be connected to the enteral connector 30.

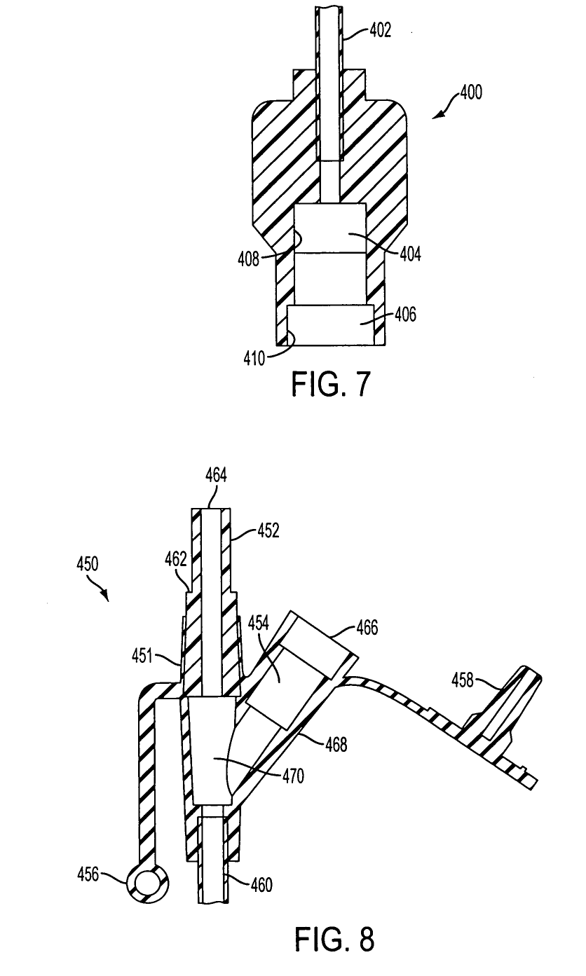

In one embodiment, the female connector 34, because of the shape of the interior port 36 and the resilient characteristics of the port 36, is configured to accept a plurality of different types and sizes of male connectors. For example, the female connector 34 may accept male connectors with different tip sizes, tapers or stepped profiles. In one embodiment, the female connector 34 has the same shape as the female connector 400 illustrated in FIG. 7.

In one embodiment, the enteral access device 62 includes a feeding tube or catheter received by the gastrointestinal tract of the patient. The catheter has an elongated tube with an end member, bolus or tip. One end of the tip includes a collar connectable to the elongated tube. The other end of the tip has a rounded or partially-rounded member. The body of the tip has a floor and a plurality of lowered side walls extending upward from the floor. The side walls define an upper elongated opening through which fluid can flow from the tube into the patient's gastrointestinal tract.

In another embodiment, the enteral access device 62 includes a feeding tube or catheter received by the gastrointestinal tract of the patient. Here, the catheter has an elongated tube. The end of the tube has a rounded or partially-rounded edge. The wall of the end region of the tube defines a plurality of holes or openings which enable enteral fluid to flow from the tube into the patient's gastrointestinal tract.

1.1.3 Multi-Port Connector

Still referring to FIG. 1, the catheter, feeding tube or enteral access device 62 includes a multi-branch connector, multi-branch or Y-port connector 46 connected to the proximal end 64 of the enteral access device 62, and the distal end 66 of the enteral access device 62 is received by the oral passage or mouth of the patient 10. Alternatively, the enteral access device 62 can enter the patient's nose. The Y-port connector 46 includes: (a) a multi-purpose branch 50; (b) a main branch 48; and (c) an internal lumen 56 enabling fluid flow. It should be appreciated that the multi-branched connector may have a Y-shape, T-shape or any other suitable shape or configuration that accommodates two or more enteral fluid sources.

The multi-purpose branch 50 of the Y-port connector 46 enables users to flush out the enteral access device 62, administer medication to the patients or conduct other enteral therapy or feeding-related activities. The multi-purpose branch 50 includes or defines a female connector 60 that is configured to receive an enteral connector 30.

The main branch 48 of the Y-port connector 46 includes or defines a male connector 80. The male connector 80 is configured to mate with the distal female connector 40 of the enteral supply tube 32. More specifically, the male connector 80 is insertable into the mating port 42 of the distal female connector 40.

In addition, the male connector 80 and the female connector 60 of the Y-port connector 46 are incompatible with or are otherwise configured to obstruct a connection with the connectors of an intravenous administration tube 18 or an intravenous supply tube 98 (e.g., male and female luer lock connectors), as described further below. Accordingly, the different profiles of the male connector 80 and the female connector 60 reduce the likelihood that the health care provider will accidentally connect the IV bag 14 or IV syringe 12 to the multi-purpose branch 50.

The Y-port connector 46 also includes two caps, plugs or stoppers 52 and 54. Stopper 52 can be used to seal and unseal the main branch 60 of the Y-port connector 46. Similarly, stopper 54 can be used to seal and unseal the multi-purpose branch 50.

It should be appreciated that the Y-port connector 46 is not required to be included in the enteral medical treatment assembly. In one embodiment, the enteral medical treatment assembly includes an enteral access device with a proximal end connected to the enteral supply tube 32 and the distal end leading to the patient. The proximal end includes a single branch defining an opening. This embodiment has the components and structure of enteral access device 62 with branch 50 removed.

In one embodiment, to install and use the enteral feeding unit or assembly 8, the user suspends the enteral feeding bag 16 from a hook 70. The hook 70 may be connected to a wall mounted unit (not shown), a pole (not shown) or any other suitable support structure. The user then inserts the enteral connector 30 of the enteral administration tube 28 into the female connector 34 of the enteral supply tube 32. Next, the user inserts the male connector 80 on the main branch 48 of Y-port connector 46 into female connector 40 of the enteral supply tube 32. The user then seals off the multi-purpose branch 50 with the stopper 54. Alternatively, the user may attach a secondary enteral fluid source to the multi-purpose branch 50. The user then primes the enteral supply tube 32 by delivering a relatively small amount of feeding fluid to the patient 10. After any occlusions have been overcome, the user administers the feeding fluid to the patient 10.

In one embodiment, the enteral connector 30 is a slightly tapered press-fit connector that is matable with female connector 34, as described above. In this embodiment, the enteral connector 30 has a leading outside diameter slightly less than the inside diameter of the mating chamber 36 of the female connector 34 female such that a user can push the male press-fit connector into the mating chamber 36 to form a fluid connection between the enteral access device 62 and the enteral supply tube 32.

1.2 Intravenous (IV) Assembly

In one embodiment, the intravenous assembly 6 includes: (a) an IV bag 14; (b) an IV administration tube 18 that connects to the IV bag 14; (c) a venous access device, venous access set or venous access assembly 22 that connects to the administration tube 18; and (d) a catheter 92 that connects to the distal end 26 of the venous access device 22 and is received by a vein or blood vessel 93 of the patient 10. It should be appreciated that the above described intravenous assembly may also function as an intra-arterial assembly where the catheter 92 is received by an artery of the patient 10.

In one embodiment, the IV bag 14 includes a plurality of volumetric markings 68 which enable the user to measure the amount of IV solution that has been administered to the patient 10, as discussed above. The IV bag 14 is supported by a hook 71, similar to the hook 70 of the enteral feeding bag 16. In operation, the IV bag 14 contains a suitable supply of IV fluid. Either gravity or a pump (not shown) forces the feeding fluid from the IV bag 14 into a drip chamber 94. The fluid passes through the drip chamber 94 into the IV supply tube or IV administration tube 18. The IV administration tube 18 includes a male luer lock connector 20 connectable to a female luer lock connector 24 at the distal end 90 of the venous access device 22. It should be appreciated that the IV assembly 6 may include any suitable type or number of intravenous administration tubing sets or pump devices that enable fluid communication from the IV bag 14 or an IV syringe 12.

In another embodiment, the intravenous assembly 6 includes: (a) an IV syringe 12; (b) an IV extension set, assembly or IV supply tube 98 that connects to the IV syringe 12; and (c) a venous access assembly or venous access device 22 that connects to the IV extension tube 98. The venous access device includes an IV catheter 92 that connects to the distal end 26 of the venous access device 22 and is received by a vein or blood vessel 93 of the patient 10. In this embodiment, the IV syringe 12 includes a male luer lock connector 97. The IV extension tube 98 includes a female luer lock connector 96 that is connectable to the male luer lock connector 97 of the IV syringe 12. The IV supply tube 98 also includes a male luer lock connector 20 that is connectable to a female luer lock connector 24 of the venous access device 22, and the female luer lock connector 24 is connected to a proximal end 90 of the venous access device 22. As mentioned above, it should be appreciated that the intravenous assembly may also function as an intra-arterial assembly where the catheter 92 is received by an artery of the patient 10. As illustrated in FIG. 1, the male luer lock connectors 97 and 20 each include: (a) an inner tube or fluid director 21 which has a conical, tapered or luer shape for a press fit connection with the female luer lock connector 24; and (b) an outer, threaded collar or lock 23 which secures each connector 97 and 20 to the female luer lock connector 24.

It should be appreciated that, in another embodiment, the intravascular assembly can include any suitable luer connectors, not necessarily the luer lock connectors 97, 20 and 24. The luer connectors of the intravascular assembly can include luer slip or luer lock connectors. In the case of luer slip connectors, each such connector includes an inner tube or fluid director which has a conical, tapered or luer shape for a press fit connection with the female luer lock connector 24. Here, the luer slip connector does not include a threaded collar or lock.

It should be understood that the intravascular assembly can include any suitable luer connector, such as any luer connector meeting the commercially available specifications of the International Organization for Standards (ISO), including, without limitation, the standards 594/1 and 594/2 for conical fittings having a designated percentage of taper.

2. Safeguard Against Erroneous Connection

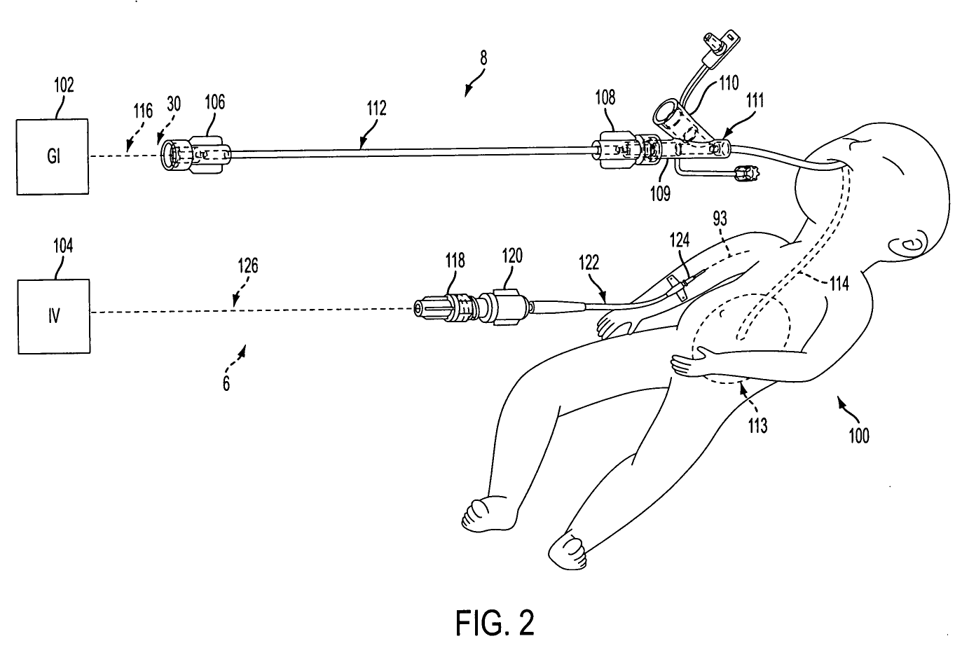

In one example illustrated in FIG. 2, the patient 100 is connected to both an enteral fluid source or supply 102 and an IV fluid source or gastrointestinal (GI) supply 104. With regard to the enteral feeding source 8: (a) the enteral connector 30 of the enteral administration tube 116 is fluidly connected with the female connector 106 of the enteral supply tube 112; (b) the female connector 108 of the enteral supply tube 112 is fluidly connected to the male connector located on the main-branch 109 of the Y-port connector 111; and (c) the enteral access device 114 is inserted into the oral or nasal cavity of the patient 100. With regard to the IV assembly 6: (a) the threaded IV male luer lock connector 118 of the IV administration tube 126 is connected to the threaded IV female luer lock connector 120 of the IV access device 122 through a screw-type connection; (b) the IV access device 122 is fluidly connected to the catheter 124; and (c) the IV catheter 124 of the IV access device 122 is inserted intravenously into the vein 93 of the patient's arm.

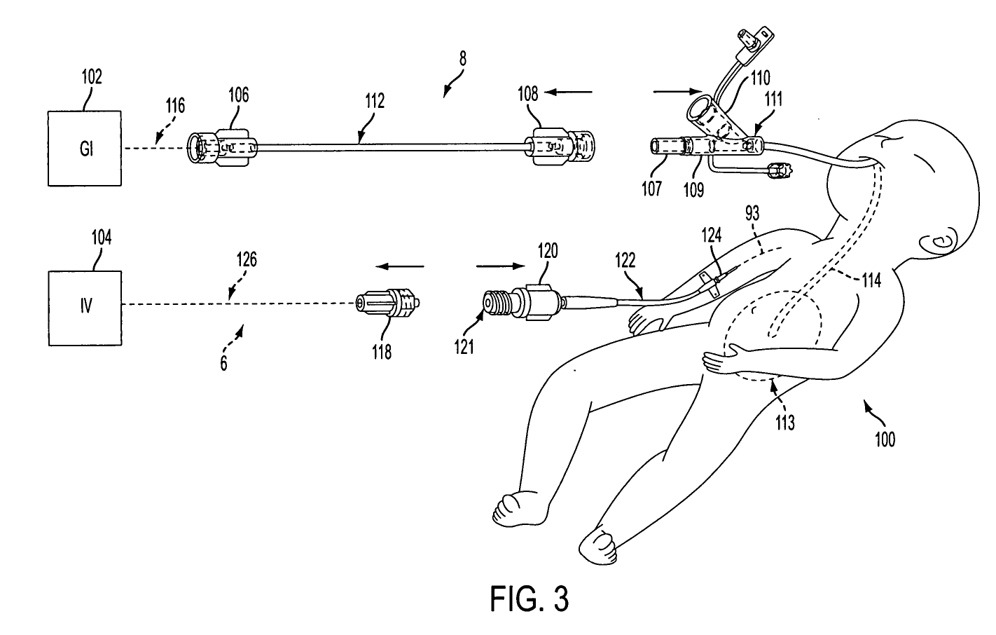

In the example illustrated in FIG. 3, the female connector 108 of the enteral supply tube 112 is disconnected from the male connector 107 located on the main-branch 109 of the Y-port connector 111. Also, the male luer lock 118 of the IV administration tube 126 is disconnected from the female luer lock 120 of the IV delivery tube 122. This may occur, for example, if a physician, nurse or other health care provider temporarily disconnects the enteral or IV assemblies to check the integrity of a connection, replace a defective part, or to substitute a fresh fluid supply. In this unconnected state, there are two fluid supply sources (specifically, the enteral fluid supply 102 and the IV fluid supply 104) and three fluid delivery input ports (specifically, the multi-purpose branch 110 and the main branch 109 of the enteral assembly, as well as the opening 121 defined by the female IV luer lock connector 120 of the IV assembly). Therefore, there is the possibility that a health care provider could cross the two systems and attempt an erroneous connection.

2.1 Attempted Erroneous Connections

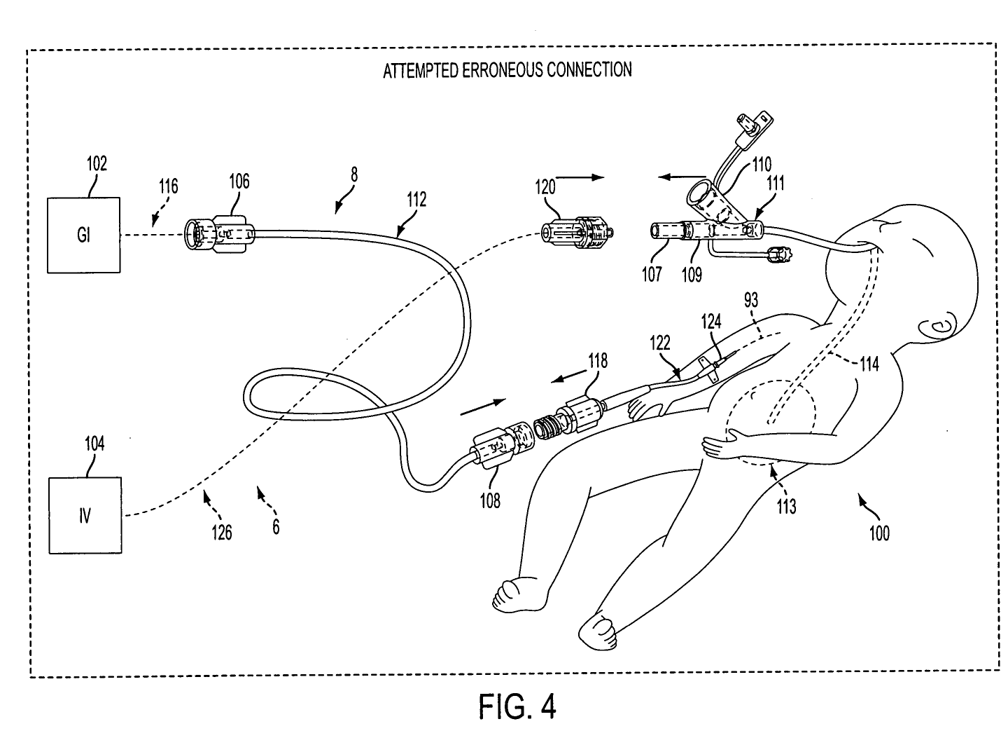

As illustrated in FIG. 4, a health care provider can erroneously attempt to connect the enteral feeding source 102 to the blood vessel 93, and the health care provider can erroneously attempt to connect the IV source 104 to the digestive tract 113. In this example, a first attempted erroneous connection occurs where the user attempts to connect the male luer lock connector 120 of the IV administration tube 126 to the male connector 107 or the multi-purpose branch 110 of the Y-port connector 111. Therefore, the user is inadvertently attempting to introduce an intravenous fluid into the gastrointestinal tract 113. A second attempted erroneous connection occurs where the user attempts to connect the female connector 108 of the enteral supply tube 112 to the female luer lock connector 118 of the IV access device 122. Here, the user is inadvertently attempting to introduce enteral fluids into the cardiovascular or circulatory system of the patient 100. Depending on the contents of the enteral fluid and the intravenous fluid, each such event could cause harm to the patient.

2.1.1 Attempted Erroneous Connection to IV Delivery Tube

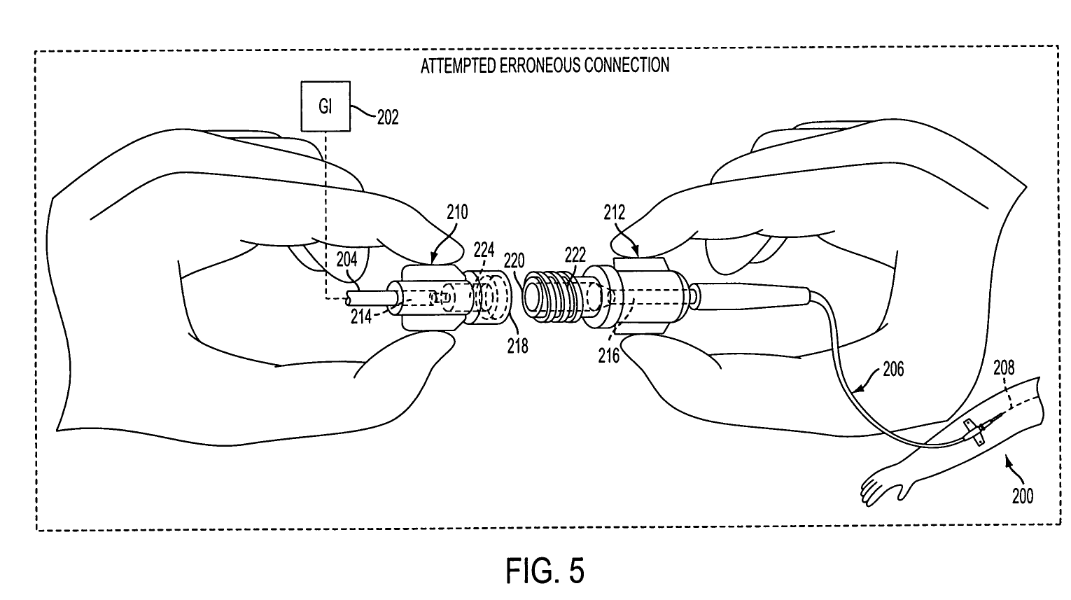

As best illustrated in FIG. 5, the physician, nurse or other health care provider may accidentally attempt to connect the female connector 210 of the enteral supply tube 204 to the female luer lock connector 212 of the IV access device 206, as described above. The health care provider is mistakenly attempting to introduce enteral fluid from the enteral fluid supply 202 into the patient's 200 circulatory system through the IV access device 206. As described above, the IV access device 206 includes a female luer lock connector 212 connector at one end of the tube, and an IV catheter 208 at the other end. In this embodiment, the female luer lock connector 212 includes an externally threaded 222 annular wall 220, and a lumen 216 to fluidly connect the luer lock connector 212 with the IV access device 206. The threads 222 enable the female luer lock connector 212 to engage and lock with the male luer lock connector 120, as described above. In this example, the female connector 210 of the enteral system 8 includes an annular wall 218 defining a mating chamber or port 224 and a lumen 214 to fluidly connect the female connecter 210 with the enteral supply tube 204.

The configuration of the enteral connector 210 causes the enteral connector 210 to be incompatible with the IV connector 212. One factor contributing to such incompatibility is that the enteral connector 210 includes an IV spacer or IV blocker. This IV blocker has a plurality of components including, without limitation: (a) a designated diameter of the enteral wall 218 which is substantially similar to the diameter of the IV wall 220; (b) a designated length of the enteral wall 218 which causes the enteral lumen 224 to recede from the end of the enteral connector 210; (c) the non-threaded and tapered configuration of the interior wall of the enteral connector 210; and (d) the female sex-type of the enteral connector 210 relative to the female sex-type of the IV connector 212. Accordingly, the enteral connector 210 is not matable with the IV connector 212. Because, in this embodiment, the enteral connector 210 is not of the luer type, the enteral connector 210 is not conveniently adaptable to be connectable to the IV connector 212. For example, in one embodiment, a user could not connect the enteral connector 210 to the IV connector 212 through use of a luer lock adapter. Accordingly, if a user attempts to connect the enteral connector 210 to the IV connector 212, the user can recognize the error before any harm may occur.

It should be appreciated that the enteral connector 210 can include any device, member or profile suitably configured to block or obstruct connection with the IV connector 212, including, but not limited to, a suitable male connector, a suitable female connector or any suitable mating differentiator or connection differentiator.

2.1.2 Attempted Erroneous Connection to Enteral Delivery Tube

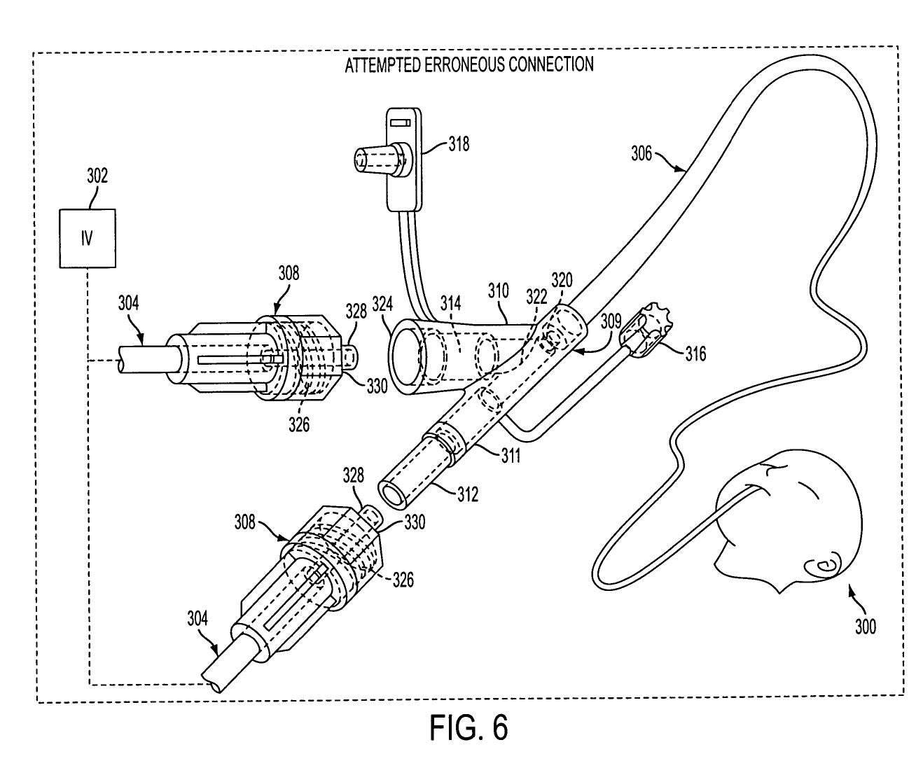

As best illustrated in FIG. 6, the user may attempt to connect the male luer lock connector 308 of the IV administration tube 304 to: (a) the female connector 314 of the multi-purpose branch 310 of the Y-port connector 309; or (b) the male connector 312 of the main branch 311 of the Y-port connector 309. Therefore, the health care provider is mistakenly attempting to introduce IV fluid from the IV fluid supply 302 into the digestive tract of the patient 300 through the enteral delivery tube 306. As described above, the enteral delivery tube 306 includes a Y-port connector 309 at one end of the tube, and the other end of the tube is received through the patient's 300 nasal or oral cavities. The Y-port connector 309 includes a main branch 311 and a multi-purpose branch 310. The main branch 311 includes a male connector 312 and plug or cap 316 to cover or seal the male connector 312. The multi-purpose branch 310 includes a female connector 314 and a plug or cap 318 to seal the annular opening 324 of the female connector 314.

The IV male luer lock connector 308 is connected to the IV administration tube 304. In this example, the male luer lock connector 308 defines two concentrically disposed annular openings. More specifically, the male luer lock 308 defines an internally threaded outer annular wall 330 and an inner annular opening 328. The outer annular opening 330 includes a sufficient number of threads 326 to properly engage the external threads of the IV female luer lock connector 212, as described above.

Due to the configuration of the enteral connectors 314 and 312, the IV male luer lock connector 308 is not matable with either the female connector 314 or the male connector 312 of the Y-port connector 309. With regard to the female connector 314, the wall 324 of the female connector 314 interferes with the internally threaded wall 326 of the IV male luer lock 308. In addition, the outside diameter of the inner annular wall 328 of the male luer lock 308 is relatively significantly less than the inside diameter of the annular opening 324 of the female connector 314. Therefore, the enteral female connector 314 functions as a blocking member or obstructer to block an erroneous connection between the IV supply 302 and the enteral delivery tube 306. With regard to the enteral male connector 312, the inner diameter of the male connector 312 is sized to deter or prevent connection with the inner annular portion 328 of the IV male luer lock connector 308. In particular, the connector 312 functions as a converter which converts the main branch 311 from a female to a male mating member. Accordingly, the connector 312 reduces the diameter of the main branch 311 to a designated diameter. Due to this designated diameter, the enteral connector 312 interferes with one or more of the IV walls 328 and 330.

If an erroneous connection were attempted, the male connector 312 would not mate with the IV male luer connector 308. Therefore, the male connector 312 functions as a blocking member or obstructer to prevent or reduce the likelihood of a misconnection between the IV supply 302 and the enteral access device 306. When the user attempts the male to male connection, the user can recognize the error before any harm may occur. It should be appreciated that the IV obstructer can include any device or member suitably configured to block or obstruct connection, including, but not limited to, an obstructer having a male shape, female shape or the shape of any suitable differentiator.

2.1.3 Proper Enteral Assembly Connection

In one embodiment illustrated in FIGS. 7 and 8, the female connector 400 defines an annular opening 404 having an inner surface 408 with a designated inside diameter and a second annular opening 406 having an inner surface 410 with a greater diameter. The Y-port connector 450 includes a main branch 451, a multi-purpose branch 468, two plugs or caps 454 and 458, a male connector 452 and a female connector 454, as described above. The main branch 451 is fluidly connected to the multi-purpose branch 468 at fluid junction 470. The male connector 452 defines an annular opening 464 and also includes a stepped portion or stepped configuration 462.

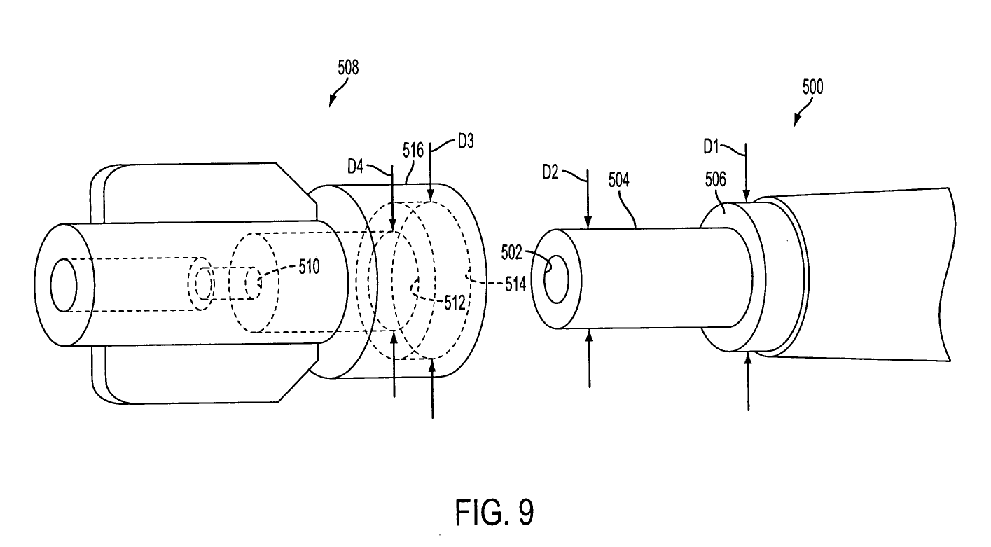

As illustrated in FIG. 9, the enteral female connector 508, in one embodiment, is configured to mate with the enteral male connector 500. The annular opening 502 of the enteral male connector 504 has an outside diameter D2 that is slightly larger than the inside diameter D4 of the first annular opening 512 of the female connector 508. As discussed above, the male connector also includes a stepped portion 506 with a diameter D1 larger than the diameter D2. Diameter D1 is slightly greater than the inside diameter D3 of the second annular opening 514 of the female connector 508. Therefore, the annular openings 512 and 514 of the female connector receive the annular opening 504 and stepped portion 506, respectively, of the male connector 500, and the connectors 500 and 508 are secured to each other through a press-fit connection.

The enteral medical treatment assembly of the present invention includes one or more intravascular or IV blocking members or obstructers integrated into the assembly. A first type of blocking member obstructs an erroneous connection between an enteral fluid source and a patient's cardiovascular or circulatory system. A second type of blocking member obstructs an erroneous connection between an intravascular or IV fluid source and a patient's digestive tract or gastrointestinal tract. Accordingly, the enteral medical treatment assembly prevents or reduces the possibility of a potential misconnection and thus, reduces health risks to the patient.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. An enteral medical treatment assembly usable adjacent to an intravascular supply tube, the intravascular supply tube having a connector connectable to an intravascular access device which is receivable by a blood vessel of a patient, the enteral medical treatment assembly comprising:
a first tube having a proximal end and a distal end, the proximal end fluidly connectable to a fluid source; and
a second tube having a proximal end and a distal end, the proximal end of the second tube connectable to the distal end of the first tube, the proximal end of the second tube having a plurality of branches, each one of the branches having a mating obstructer configured to obstruct mating of each one of said branches with the connector of the intravascular supply tube.

2. The enteral medical treatment assembly of claim 1, wherein the distal end of the first tube includes a female connector.

3. The enteral medical treatment assembly of claim 2, wherein the plurality of branches include a first branch having a male connector and a second branch having a female connector, wherein the male connector and the female connector each include an intravascular mating obstructer.

4. The enteral medical treatment assembly of claim 3, wherein the male connector of the second tube is matably insertable into the female connector of the first tube.

5. The enteral medical treatment assembly of claim 4, wherein the male connector of the second tube has a marking characteristic in common with the female connector of the first tube.

6. The enteral medical treatment assembly of claim 5, wherein the marking characteristic includes a characteristic selected from the group consisting of a color, a symbol, an image, text and a code.

7. An enteral medical treatment assembly usable adjacent to an intravascular medical treatment system having an intravascular source and an intravascular supply tube, the intravascular supply tube having a connector connectable to an intravascular access device which is receivable by a blood vessel of a patient, the enteral medical treatment assembly comprising:
a first tube having a proximal end and a distal end, the proximal end of the first tube fluidly connected to a fluid source, and the second end of the first tube including a female connector, and
a second tube having a first end and a second end, the first end having a multi-port connector, the multi-port connector including a primary branch and a secondary branch, the primary branch configured to obstruct mating of said branch with the connector of the intravascular supply tube or any portion of the intravascular source.

8. The enteral medical treatment assembly of claim 7, wherein the primary mating blocker includes a male connector, the male connector matably insertable into the female connector of the first tube.

9. The enteral medical treatment assembly of claim 8, wherein the connector of the intravascular supply tube includes a male luer connector.

10. The enteral medical treatment assembly of claim 8, wherein the male connector of the second tube has a marking characteristic in common with the female connector of the first tube.

11. The enteral medical treatment assembly of claim 10, wherein the marking characteristic includes a characteristic selected from the group consisting of a color, a symbol, an image, text and a code.

12. The enteral medical treatment assembly of claim 7, wherein the secondary branch is configured to obstruct mating of said branch with the connector of the intravascular supply tube, wherein the secondary branch has a different configuration than the primary branch.

13. The enteral medical treatment assembly of claim 12, wherein the secondary branch includes a female connector.

14. An enteral medical treatment assembly usable adjacent to an intravascular supply tube, the intravascular supply tube having a connector connectable to an intravascular access device which is receivable by a blood vessel of a patient, the enteral medical treatment assembly comprising:
a first tube having a proximal end and a distal end, the proximal end of the first tube fluidly connected to a fluid source, the distal end of the first tube including a female connector; and
a second tube having a proximal end and a distal end, the proximal end including a male connector, the male connector matably insertable into the female connector of the first tube, the male connector configured to block mating of the proximal end of the second tube with the connector of the intravenous supply tube.

15. The enteral medical treatment assembly of claim 14, wherein the connector of the intravascular supply tube includes a male connector.

16. The enteral medical treatment assembly of claim 15, wherein the female connector of the first tube is configured to block mating of the first tube with a female connector of the intravascular access device.

17. The enteral medical treatment assembly of claim 14, wherein the distal end of the second tube is received, directly or indirectly, by a portion of a digestive tract of a patient.

18. An enteral medical treatment assembly usable adjacent to an intravascular access device, the intravascular access device having: (a) a distal end receivable by a blood vessel of a patient; and (b) a proximal end positionable external to the patient, the proximal end having a connector connectable to a portion of an intravascular treatment system, the enteral medical treatment assembly comprising:
a first tube having a proximal end and a distal end, the proximal end of the first tube fluidly connectable to a fluid source, the distal end of the first tube including a connector configured to block mating of the first tube with the connector of the intravascular access device; and
a second tube having a proximal end and a distal end, the proximal end of the second tube connectable to the distal end of the first tube, the second tube defining at least one fluid outlet adjacent to the distal end of the second tube.

19. The enteral medical treatment assembly of claim 18, wherein the proximal end of the second tube is configured to block mating of the second tube with a portion of the intravascular treatment system.

20. The enteral medical treatment assembly of claim 19, wherein the connector of the first tube has a designated sex type, and the connector of the intravascular access device has an identical sex type.

21. The enteral medical treatment assembly of claim 19, wherein the connector of the first tube has at least one wall which is sized and configured to obstruct mating of the connector of the first tube with the connector of the intravascular access device.

22. An enteral medical treatment assembly usable adjacent to an intravascular access device, the intravascular access device having: (a) a distal end receivable by a blood vessel of a patient; and (b) a proximal end positionable external to the patient, the proximal end having a connector, the enteral medical treatment assembly comprising:
(a) a first enteral tube having:
(i) a proximal end fluidly connectable to a fluid source; and
(ii) a distal end having a mating obstructer configured to obstruct mating of the distal end of the first enteral tube with the connector of the intravascular access device; and
(b) a second enteral tube having:
(i) a proximal end;
(ii) a distal end receivable by a portion of a gastrointestinal tract of the patient; and
(iii) a wall adjacent to the distal end, the wall defining at least one fluid outlet.

23. The enteral medical treatment assembly of claim 22, wherein the proximal end of the second tube is configured to obstruct connection of said tube with the connector of a portion of an intravascular treatment system.

24. The enteral medical treatment assembly of claim 22, wherein the proximal end of the second enteral tube is configured to obstruct mating of the second enteral tube with the connector of a portion of an intravascular treatment system.

25. The enteral medical treatment assembly of claim 24, which includes a multi-branch connector connected to the proximal end of the second enteral tube.

26. The enteral medical treatment assembly of claim 22, wherein the distal end of the second enteral tube includes a tip, the tip having a body, a collar and a partially-rounded end member, the body defining an upper fluid outlet positioned between the collar and the partially-rounded end member.

27. The enteral medical treatment assembly of claim 22, wherein the distal end of the second enteral tube includes a end region having a wall and a tip, the wall defining a plurality of fluid outlets, and the tip having a partially rounded edge.

28. An enteral medical treatment assembly usable adjacent to an intravascular medical treatment system, the enteral medical treatment assembly comprising:
a first tube having a proximal end and a distal end, the proximal end fluidly connectable to a fluid source; and
a second tube having a proximal end and a distal end, the proximal end of the second tube having a mating obstructer configured to obstruct mating of the second tube with any portion of the intravascular medical treatment system.

29. The enteral medical treatment assembly of claim 28, wherein the mating obstructer is configured to obstruct mating with any connector of an intravascular system.

30. The enteral medical treatment assembly of claim 28, wherein the mating obstructer is configured to obstruct mating with a luer connector of an intravascular supply tube.

31. The enteral medical treatment assembly of claim 28, wherein the mating obstructer includes a rigid size reducer.
